(19) United States
(12) Patent Application Publication
(10) Pub. No.: US 2009/0240260 A1
(43) Pub. Date: Sep. 24, 2009

(54) HAIR REMOVAL DEVICE

(75) Inventors: Zheng Qing Yang, Kwung Tong (HK); Jin Ye, Guangzhou (CN)

Correspondence Address: LEYDIG VOIT & MAYER, LTD, 700 THIRTEENTH ST. NW, SUITE 300, WASHINGTON, DC, 20005-3960, US

(73) Assignee: Ever Shine Technology Limited, Kowloon (HK)

(21) Appl. No.: 12/341,173

(22) Filed: Dec. 22, 2008

(30) Foreign Application Priority Data
Mar. 19, 2008 (DE) 10 2008 014 946.2

(51) Int. Cl. A61B 17/50; A45D 24/10

(52) U.S. Cl. 606/133; 132/148

(57) ABSTRACT

A hair-plucking assembly includes a helical springs motor-driven to rotate about a straight axis. Cam devices displace opposing ends of the spring periodically as the spring rotates, opening spaces between adjacent loops of the spring to receive hair. The loops are then closed to pinch and pluck the hair. A combing device includes a quasi-continous longitudinal channel in the imaginary outer surface of the spring.

### TECHNICAL FIELD

The present invention relates to motorised epilating devices for removing unwanted body hair by uprooting the hair.

### BACKGROUND OF THE INVENTION

A known epilating device, such as that described in U.S. Pat. No. 4,935,024, includes a motor-driven rotating helical spring bent into an arcuate configuration to produce spaces between adjacent loops of the spring on a convex side. Hair received in these spaces is pinched between adjacent loops and plucked when these loops close together as the spring rotates toward the concave side.

While these prior art epilating devices perform satisfactorily, it will be understood that in developing epilating devices of this type, care must be taken to ensure that the pulling of hair occurs sufficiently quickly that the user does not sense an unacceptable amount of discomfort. To achieve this it is also preferable that there be a relatively large number of hair-plucking movements—opening and closing the hair-receiving space—with each rotation of the spring. To grasp the hair and uproot it, and avoid cutting of the hair, it is desirable to increase the area of contact between which hairs are grasped. Of course, in addressing these requirements it is important that the product can be produced cost-effectively, having components which are not unnecessarily complicated or otherwise costly to manufacture, while also allowing for ready assembly and maintenance of the unit. It is an object of the present invention to meet the above needs or more generally to provide an improved epilating device.

### DISCLOSURE OF THE INVENTION

According to one aspect of the present invention there is provided an epilating device, comprising:

a housing;

a spring supported in the housing for rotation about a straight axis, the spring being helical, adjacent loops of the spring defining spaces for receiving and pinching hair therebetween;

a rotary motor disposed in the housing and drivingly connected to the helical spring, and

at least one cam and cam-follower pair, relative rotation between the cam and cam-follower displacing an end of the spring axially and closing the spaces periodically as the spring rotates to pluck the hair.

Preferably one of the cam and cam-follower of the at least one pair is fixed to the housing, the other of the cam and cam-follower of the at least one pair is fixed to an end of the spring, and the spring urges the cam and cam-follower into engagement. Preferably the spring is maintained in compression. Optionally, the spring may be maintained in tension.

Preferably the device comprises first and second cam and cam-follower pairs, each pair engaging an opposing end of the spring and both pairs cooperating to substantially simultaneously displace both ends of the spring axially and close the spaces periodically as the spring rotates.

Preferably the cam has a cam surface extending circumferentially relative to the axis and the cam-follower has at least one contacting face making sliding contact with the cam surface.

Preferably the cam surface includes diametrically opposing concave portions and the cam-follower has a two contacting faces disposed diametrically opposite one another. Preferably the contacting faces are spheroidal.

Preferably the cam and cam-follower pair displace the spring to open and close the spaces at least twice per revolution of the spring.

Preferably the spring is formed from a thread and includes a combing device to align the hair to be pinched in the spaces and plucked out, the combing device comprising at least one groove extending substantially transversely to the thread and lying in the area of an imaginary outer surface of the spring in at least one loop of the spring.

Preferably adjacent loops of the spring are each provided with at least one groove, the grooves forming a quasi-continuous channel in the area of the imaginary outer surface of the spring.

Preferably the channel extends parallel to the axis of the spring. Preferably the channels have substantially identical cross-sections.

Preferably the groove has a cross-section with a first flat thereon, the first flat defining a radially outermost surface of the spring. Preferably the thread comprises awire.

Preferably the imaginary outer surface of the spring is substantially cylindrical. Optionally imaginary outer surface may be convex or barrel-shaped. Alternatively the imaginary outer surface may be concave or waisted in shape.

This invention provides an epilating device which is effective and efficient in operational use, providing reliable clamping action over a large area between the adjacent loops of the spring. In particular, the device may be economically constructed and has an overall simple design which minimizes manufacturing costs and increases the number of common parts that are used.

### BRIEF DESCRIPTION OF THE DRAWINGS

Preferred forms of the present invention will now be described by way of example with reference to the accompanying drawings, wherein:

FIG. 1a is side view of an epilating device according to the present invention;

FIG. 1b is side view of the epilating device of FIG. 1a showing part of the housing removed;

FIG. 2 is an exploded perspective view of the hair plucking assembly of the device of FIG. 1a;

FIG. 3 is a fragmentary side view of the spring of the hair plucking assembly of FIG. 2;

FIG. 4 is a fragmentary end view of spring of FIG. 1a;

FIG. 5 is a fragmentary sectional view along line AA of FIG. 3;

FIG. 6 is a central longitudinal section through the hair plucking assembly of the device of FIG. 1a in a first position, and

FIG. 7 is a view corresponding to FIG. 6 showing the cam follower rotated 90 degrees.

### DESCRIPTION OF THE PREFERRED EMBODIMENTS

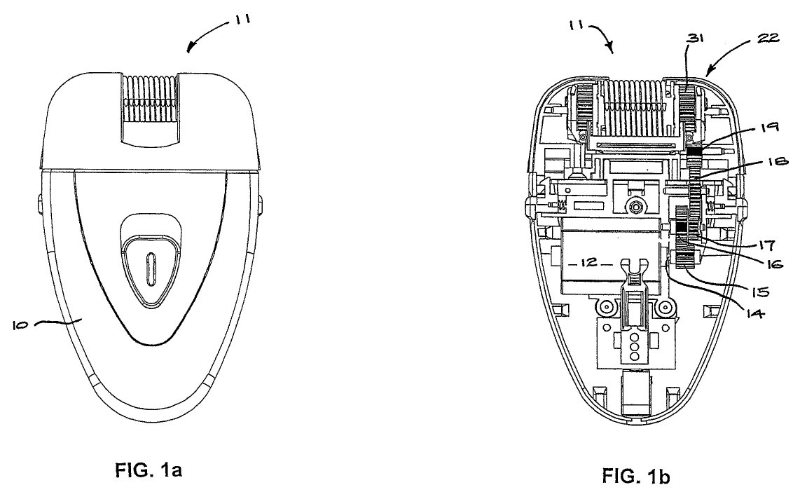

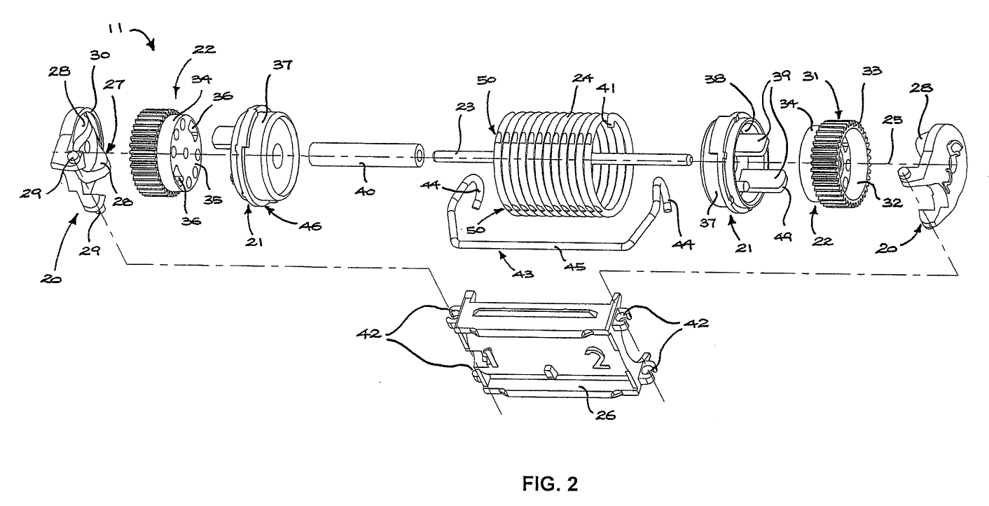

Referring to the drawings, FIGS. 1 and 2 illustrate a preferred embodiment of an epilating device having a housing 10, or handle, which is manually grippable by the user; and a hair-plucking assembly 11. The housing 10 holds a motor 12 powered by mains power through a plug connector 13, although in alternative embodiments it may be powered by batteries.

A reduction gear train serves to transmit torque from the motor output to the hair-plucking assembly 11 which is rotated at a reduced speed. In the system illustrated in FIG. 1, a pinion 15 is mounted on the motor shaft 14 and meshes with a gear 16 mounted rotationally fast with gear 17 to provide a first reduction. Gear 17 engages with a gear 18 to provide a second reduction. An idler gear 19 transfers torque from the gear 18 to a journal block 22, engaging a toothed gear portion 31 thereof.

Hair-plucking assembly 11 includes a cam device mounted at each end of the spring 24 and further includes a cam 20, a cam-follower 21 and a journal block 22 each with apertures therethrough for receiving an axle 23. As used herein, the term “axial” refers to a direction substantially parallel to the longitudinal axis of the assembly and the term “radial” refers to a direction substantially orthogonal to this axes. The term “circumferential” refers to the direction of a circular arc having a radius substantially orthogonal to the axis 25.

Each cam 20 includes a circumferentially-extending cam surface 27 which is generally symmetrical and includes two diametrically opposing lobes 28. The cam surface 27 is partly surrounded by a larger diameter abutment face 30 which lies in a transverse plane. The cam 20 further includes coaxial cylindrical tabs 29 integral therewith.

Each journal block 22 includes a gear portion 31 having a plurality of external teeth. Opening outwardly is a recess 32 in which the lobes 28 of cam 20 are received and which is surrounded with a rim 33 that generally abuts the abutment face 30. Extending axiallty inwardly of the gear portion 31 is a cylindrical face 34. An inner transverse face 35 includes a pair of axially-extending openings 36 in diametrically opposite positions.

Each cam-follower 21 includes a spigot portion 37 with a cylindrical face and a concavity 38 with inner walls complementary to the cylindrical face 34 received therein. The spigot portion 37 is bounded by a radially-outwardly-projecting shoulder 46. Projecting axially from the concavity 38 are two parallel arms 39 located diametrically opposite one another and sized for a sliding fit in respective ones of the openings 36 such that the cam follower 22 is thereby rotated together with the journal block 22. The arms 39 are curved in cross-section, with coaxial concave and convex faces on their radially inner and outer sides respectively.

The spring 24 is helically wound from wire thread and has a shape defining imaginary inner and outer cylindrical boundary surfaces, the axially opposing ends of the spring 24 being received and supported by the spigot portions 37 and abutting the shoulders 46. Opposing ends 41 of the wire from which the spring is wound project inwardly through the imaginary inner boundary surface of the spring and are received in apertures in the cam-followers 21, so that the spring 24 is rotated together with the cam-followers 21. The axle is received in a tubular spacer 40, the opposing ends of which abut the two cam-followers 21.

A bracket 26 includes a pairs of recesses 42 at its opposing ends for receiving the tabs 29, to mount the cams 20. A resilient metal clip 43 has an elongate portion 45 which abuts the bracket 26 and hooks 44 at its ends which engage the opposite ends of the axle 23. When assembled the spring 24 urges the convex ends 49 of the arms 39 against the cam surface 37.

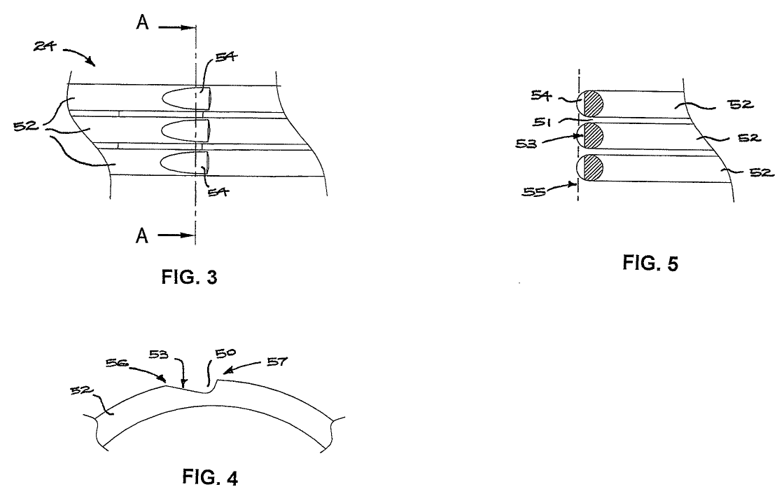

FIGS. 2-5 illustrate in detail one of four quasi-continuous channels 50 which are formed in the outer surface of the spring 24. The channels 50 are elongate axially and substantially equally angularly spaced. The channels 50 form a combing device to align the hair to be pinched in the spaces 51 between adjacent loops 52 of the spring.

Each channel 50 is quasi-continuous in as much as it is defined by a groove 54 in each of the loops 52. Each groove 54 extends substantially transversely to the wire and lies recessed below an imaginary cylindrical outer surface 55 of the spring. The spring 24 is wound from wire having a circular cross-section and the grooves 54 are formed as by grinding. Each groove 54 has a shape which is asymmetrical in a transverse direction, having a surface 53 with a leading edge 56 and trailing edge 57 which intersect at a relatively small angle, and a relatively large angle respectively, to the convex face of the wire.

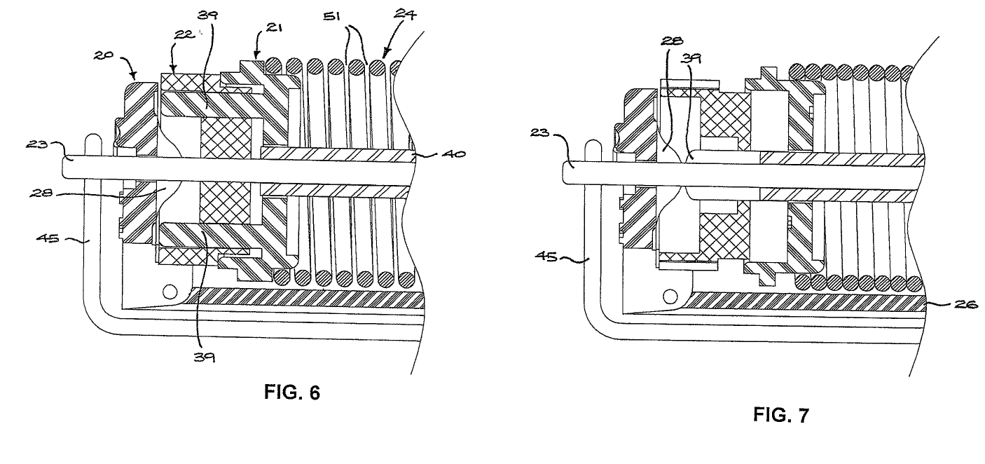

The operation of the hair-plucking assembly 11 is best seen with respect to FIGS. 6 and 7, where the spring is shown expanded and compressed respectively. With the spring expanded to open the hair-receiving spaces 51 between loops 52 the arms 39 are positioned 900 out of phase with the lobes 28 (FIG. 6). Relative rotation between the cam and cam-follower thereafter impels both of the cam-followers 21 axially, moving them together until the arms 39 abut the crests of respective lobes 28 whereupon the spring is fully compressed to generally close the spaces between the loops (FIG. 7).

It will be understood that throughout the operation the axis of the spring 24 remains straight and the outermost ends of loops of the spring are displaced axially without rotation. The spring 7 is compressed twice and relaxed twice per revolution, thereby providing a relatively large number of hair-plucking movements per rotation.

1. An epilating device, comprising:
a housing;
a spring supported in the housing for rotation about a straight axis, the spring being helical, adjacent loops of the spring defining spaces for receiving and pinching hair the loops:
a rotary motor disposed in the housing and drivingly connected to the helical spring; and
at least one cam and cam-follower pair, relative rotation between the cam and cam-follower displacing an end of the spring axially and closing the spaces periodically, as the spring rotates, to pluck the hair.

2. The epilating device of claim 1 wherein one of the cam and cam-follower of the at least one pair is fixed to the housing, the other of the cam and cam-follower of the at least one pair is fixed to an end of the spring, and the spring urges the cam and cam-follower into engagement.

3. The epilating device of claim 2 wherein the spring is maintained in compression.

4. The epilating device of claim 3 wherein the device comprises first and second cam and cam-follower pairs, each pair engaging a respective end of opposing ends of the spring and both pairs cooperating to substantially simultaneously displace both ends of the spring axially and close the spaces periodically, as the spring rotates.

5. The epilating device of claim 1 wherein the cam has a cam surface extending circumferentially relative to the axis and the cam-follower has at least one contacting face making sliding contact with the cam surface.

6. The epilating device of claim 5 wherein the cam surface includes diametrically opposing concave portions and the cam-follower has a two contacting faces disposed diametrically opposite one another.

7. The epilating device of claim 6 wherein the contacting faces are spheroidal.

8. The epilating device of claim 6 wherein the cam and cam-follower pair displace the spring to open and close the spaces at least twice per revolution of the spring.

9. The epilating device of claim 1 wherein the spring includes a thread and a combing device to align the hair to be pinched in the spaces and plucked out, the combing device comprising at least one groove extending substantially transverse to the thread and lying in an imaginary outer surface of the spring in at least one loop of the spring.

10. The epilating device of claim 9 wherein adjacent loops of the spring each include at least one groove, the grooves forming a quasi-continuous channel in the imaginary outer surface of the spring.

11. The epilating device of claim 10 wherein the channel extends parallel to the axis of the spring.

12. The epilating device of claim 1 including two or more channels extending parallel to one another in the imaginary outer surface of the spring.

13. The epilating device of claim 12 wherein each of the channels has a substantially identical cross-section, each groove having a shape which is asymmetrical in a transverse direction, having a surface with a leading edge and trailing edge which respectively intersects at a relatively small angle, and a relatively large angle a convex face of the wire.

14. The epilating device of claim 1 wherein
the spring includes a thread, and
the thread has a cross-section with a first flat, the first flat defining a radially outermost surface of the spring.

15. The epilating device of claim 14 wherein the thread further includes a second flat, the second flat defining a radially innermost surface of the spring.

16. The epilating device of claim 14 wherein the thread comprises a wire.

17. The epilating device of claim 16 wherein the imaginary outer surface of the spring is substantially cylindrical.

18. An epilating device, comprising:
a housing;
a spring supported in the housing for rotation about a straight axis, the spring being helical, adjacent loops of the spring defining spaces for receiving and pinching hair the loops;
a rotary motor disposed in the housing and drivingly connected to the helical spring, and
first and second cam and cam-follower pairs, each pair engaging a respective one of opposing ends of the spring, one of the cam and cam-follower of the each pair being fixed to the housing, the other of the cam and cam-follower of each pair being fixed to the spring, wherein
the spring urges the cam and cam-follower of each pair into engagement and both pairs cooperating to substantially simultaneously displace both ends of the spring axially and close the spaces periodically, as the spring rotates,
each cam has a cam surface extending circumferentially relative to the axis, and
each cam-follower has at least one contacting face making sliding contact with the cam surface.

19. The epilating device of claim 18 wherein each cam surface includes diametrically opposing concave portions and the cam-follower has a two contacting faces disposed diametrically opposite one another.

20. The epilating device of claim 19 wherein the cam and cam-follower pair displace the spring to open and close the spaces at least twice per revolution of the spring.

21. The epilating device of claim 20 wherein the spring includes a thread and a combing device to align the hair to be pinched in the spaces and plucked out, the combing device comprising at least one groove extending substantially transverse to the thread and lying in an imaginary outer surface of the spring in at least one loop of the spring.

22. The epilating device of claim 21 wherein adjacent loops of the spring each include at least one groove, the grooves forming a quasi-continuous channel in the imaginary outer surface of the spring.

23. The epilating device of claim 22 wherein the channel extends parallel to the axis of the spring.

24. The epilating device of claim 23 including two or more channels extending parallel with to one another in the imaginary outer surface of the spring.

25. The epilating device of claim 23 wherein each of the channels has a substantially identical cross-section, each groove having a shape which is asymmetrical in a transverse direction, having a surface with a leading edge and trailing edge which respectively intersects at a relatively small angle, and a relatively large angle a convex face of the wire.

26. The epilating device of claim 18 wherein
the spring includes a thread, and
the thread has a cross-section with a first flat, the first flat defining a radially outermost surface of the spring.

27. The epilating device of claim 26 wherein the thread further includes a second flat, the second flat defining a radially innermost surface of the spring.

28. The epilating device of claim 26 wherein the thread comprises a wire.

29. The epilating device of claim 26 wherein the imaginary outer surface of the spring is substantially cylindrical.
